(19) United States
(12) Patent Application Publication
(10) Pub. No.: US 2025/0352605 A1
(43) Pub. Date: Nov. 20, 2025

(54) DIETARY SUPPLEMENTS FOR MOOD SUPPORT

(71) Applicant: Melaleuca, Inc., Idaho Falls, ID (US)

(72) Inventors: Nasser A. Fredj, Idaho Falls, ID (US); Jordan Joy, Idaho Falls, ID (US); Alexander B. Rabovsky, Idaho Falls, ID (US)

(21) Appl. No.: 19/207,947

(22) Filed: May 14, 2025

Related U.S. Application Data
(60) Provisional application No. 63/647,909, filed on May 15, 2024.

(51) Int. Cl. A61K 36/88; A61K 31/519; A61K 31/714; A61K 36/81

(52) U.S. Cl. CPC A61K 36/88; A61K 31/519; A61K 31/714; A61K 36/81

(57) ABSTRACT

Mood support compositions are described herein. For example, this document provides mood support compositions containing saffron, ashwagandha, and vitamin B12. Such compositions may be used to, e.g., support a positive mental outlook, promote emotional health, support general well-being, and reduce feelings of occasional stress.

### CROSS-REFERENCE TO RELATED APPLICATION

This application claims the benefit of U.S. Provisional Application No. 63/647,909, filed on May 15, 2024, the disclosure of which is incorporated herein by reference in its entirety.

### TECHNICAL FIELD

This document relates to the field of mood support compositions. For example, this document relates to mood support compositions useful for one or more of supporting a positive mental outlook, promoting emotional health, supporting general well-being, and reducing feelings of occasional stress.

### BACKGROUND

Mental health issues are a very real brain health concern. Neurotransmitters such as serotonin can affect mood and one's ability to cope with stress. Proper vitamins and supplementation can support the brain as it functions throughout life and help the produce the proper amounts of necessary chemicals like serotonin. If the brain is receiving proper nutrients, it can start to send signals that something is wrong. For example, some of the signals the brain can send are anxiousness, feelings of being overwhelmed, or unshakable feeling of sadness. There is a continuing need to formulate compositions that support and/or improve mood.

### SUMMARY

Provided herein are mood support compositions comprising: a saffron extract; an ashwagandha extract; folate; and vitamin B12, wherein the vitamin B12 comprises methylcobalamin.

In some embodiments, the saffron extract is present in an amount of about 14 mg to about 56 mg per serving of the mood support composition. In some embodiments, the saffron extract is present in an amount of about 28 mg to about 30 mg per serving of the mood support composition. In some embodiments, the saffron extract is present in an amount of about 28 mg per serving of the mood support composition.

In some embodiments, the saffron extract is an extract of the stigmas of Crocus sativus flowers.

In some embodiments, the ashwagandha extract is present in an amount of about 125 mg to about 1800 mg per serving of the mood support composition. In some embodiments, the ashwagandha extract is present in an amount of about 300 mg to about 675 mg per serving of the mood support composition. In some embodiments, the ashwagandha extract is present in an amount of about 600 mg per serving of the mood support composition.

In some embodiments, the ashwagandha extract contains one or more withanolides.

In some embodiments, the ashwagandha extract is an ashwagandha root extract.

In some embodiments, vitamin B12 is present in an amount of about 0.5 μg to about 100 μg per serving of the mood support composition. In some embodiments, vitamin B12 is present in an amount of about 3 μg to about 60 μg per serving of the mood support composition. In some embodiments, vitamin B12 is present in an amount of about 6 μg per serving of the mood support composition.

In some embodiments, the folate is present in an amount of about 0.1 mg to about 2 mg DFE per serving of the mood support composition. In some embodiments, the folate is present in an amount of about 0.2 mg to about 0.8 mg DFE per serving of the mood support composition. In some embodiments, the folate is present in an amount of about 0.4 mg DFE per serving of the mood support composition.

In some embodiments, the folate comprises calcium methyltetrahydrofolate.

In some embodiments, the mood support composition is formulated as a tablet, a capsule, or a soft gel. In some embodiments, two tablets, capsules, or soft gels comprises a full serving of the mood support composition. In some embodiments, the mood support composition is formulated as a capsule.

Also provided herein are methods of improving one or more of emotional health, the ability to cope with stress, the activity of a brain messenger, emotional balance, general well-being, the restfulness of sleep, physical energy, and mental energy in a mammal, comprising administering to the mammal any of the mood support compositions described herein.

Also provided herein are methods of reducing feelings of sadness and/or feelings of stress in a mammal, comprising administering to the mammal any of the mood support compositions described herein.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DETAILED DESCRIPTION

This document provides mood support compositions that contain saffron (e.g., a saffron extract), ashwagandha (e.g., an ashwagandha extract), and vitamin B12. Such mood support compositions can be useful for one or more of supporting a positive mental outlook, better coping with the ups and downs of life, maintaining a balanced mood, promoting emotional health, supporting emotional balance, supporting the activities of brain messengers, supporting general well-being, supporting more restful sleep (e.g., restful sleep without sedation), reducing feelings of increased sadness, reducing feelings of occasional stress, supporting mood-related sexual health, and promoting physical and mental energy (e.g., after consuming a mood support composition provided herein as compared to not taking a mood support composition provided herein).

### Compositions

### Saffron

The mood support compositions provided herein include saffron. Saffron is a spice derived from the stigmas of Crocus sativus flowers. Saffron can increase the levels of mood-elevating neurotransmitters like serotonin and dopamine. Its ability to modulate neurotransmitters like serotonin, dopamine, and norepinephrine may contribute to its effects on mood. Studies on saffron have shown comparable efficacy to common selective serotonin reuptake inhibitors in reducing depression and anxiety. For improving sleep, studies have shown that saffron can have a beneficial effect. Additionally, saffron has been reported to have antioxidant and anti-inflammatory properties.

Saffron, as used herein, can include saffron threads (e.g., dried whole stigmas), powdered saffron (e.g., ground stigmas), and/or a saffron extract. A saffron extract can be in the form of a liquid (e.g., an aqueous solution), a solid (e.g., a particulate or a powder), or a semi-solid form (e.g., a gel or an emulsion). Any appropriate method can be used to obtain a saffron extract for use in the mood support compositions provided herein. For example, a saffron extract can be obtained by aqueous or ethanolic extraction from Crocus sativus stigma.

The mood support compositions provided herein can contain any appropriate amount of saffron. In some embodiments, saffron is present in an amount of at least about 10 mg per serving of a mood support composition provided herein (e.g., at least about 14 mg, at least about 20 mg, at least about 24 mg, at least about 28 mg, at least about 32 mg, at least about 36 mg, at least about 44 mg, or at least about 50 mg per serving of a mood support composition provided herein). In some embodiments, saffron is present in an amount of no more than about 60 mg per serving of a mood support composition provided herein (e.g., no more than about 50 mg, no more than about 44 mg, no more than about 36 mg, no more than about 32 mg, no more than about 28 mg, or no more than about 24 mg per serving of a mood support composition provided herein).

In some embodiments, a saffron extract is present in an amount of about 14 mg to about 56 mg per serving of a mood support composition provided herein. For example, a saffron extract can be present in an amount of about 14 mg to about 20 mg, about 14 mg to about 26 mg, about 14 mg to about 32 mg, about 14 mg to about 38 mg, about 14 mg to about 44 mg, about 14 mg to about 50 mg, about 20 mg to about 26 mg, about 20 mg to about 32 mg, about 20 mg to about 38 mg, about 20 mg to about 44 mg, about 20 mg to about 50 mg, about 20 mg to about 56 mg, about 32 mg to about 38 mg, about 32 mg to about 44 mg, about 32 mg to about 50 mg, about 32 mg to about 56 mg, about 38 mg to about 44 mg, about 38 mg to about 50 mg, about 38 mg to about 56 mg, about 44 mg to about 50 mg, about 44 mg to about 56 mg, or about 50 mg to about 56 mg per serving of a mood support composition provided herein. In some embodiments, a saffron extract is present in an amount of about 24 mg to about 32 mg per serving of a mood support composition provided herein. In some embodiments, a saffron extract is present in an amount of about 26 mg to about 30 mg per serving of a mood support composition provided herein. In some embodiments, a saffron extract is present in an amount of about 28 mg to about 30 mg per serving of a mood support composition provided herein.

In some embodiments, a saffron extract is present in an amount of about 14 mg, 20 mg, about 22 mg, about 24 mg, about 26 mg, about 28 mg, about 30 mg, about 32 mg, about 34 mg, about 36 mg, about 38 mg, about 44 mg, about 50 mg, or about 56 mg per serving of a mood support composition provided herein. In some embodiments, a saffron extract is present in an amount of about 28 mg per serving of a mood support composition provided herein.

In some embodiments, a saffron extract is present in an amount of about 1% to about 30% w/w of a mood support composition provided herein. For example, a saffron extract can be present in an amount of about 1% to about 5%, about 1% to about 10%, about 1% to about 15%, about 1% to about 20%, about 1% to about 25%, about 5% to about 10%, about 5% to about 15%, about 5% to about 20%, about 5% to about 25%, about 5% to about 30%, about 10% to about 15%, about 10% to about 20%, about 10% to about 25%, about 10% to about 30%, about 15% to about 20%, about 15% to about 25%, about 15% to about 30%, about 20% to about 25%, about 20% to about 30%, or about 25% to about 30% w/w of a mood support composition provided herein. In some embodiments, a saffron extract is present in an amount of about 1%, about 5%, about 10%, about 15%, about 20%, about 25%, or about 30% w/w of a mood support composition provided herein.

### Ashwagandha

The mood support compositions provided herein include ashwagandha. Ashwagandha (Withania somnifera) is an evergreen shrub found in tropical and subtropical areas of Asia, Africa, and Europe. It has been traditionally used for reducing stress. Ashwagandha is an adaptogen that can help the body adapt to stress and boost energy, stamina, and endurance. It can support the body's ability to cope with stress by modulating the stress response system and promoting a sense of calm. For example, Ashwagandha can have cortisol-lowering effects and anti-inflammatory properties to help adapt to change and stress. Ashwagandha can also support cognitive function, promote relaxation, and improve sleep quality.

In some embodiments, the mood support compositions provided herein can include ashwagandha whole root powder and/or an ashwagandha extract. An ashwagandha extract can be in the form of a liquid (e.g., an aqueous solution), a solid (e.g., a particulate or a powder), or a semi-solid form (e.g., a gel or an emulsion). Any appropriate method can be used to obtain ashwagandha for use in the mood support compositions provided herein. For example, ashwagandha whole root powder can be prepared by drying and grinding the root of the ashwagandha plant. As another example, an alcohol (e.g., ethanol) solution can be used to obtain an ashwagandha extract from ashwagandha roots and/or ashwagandha leaves.

In some embodiments, a mood support composition as provided herein includes ashwagandha whole root powder. The mood support compositions provided herein can contain any appropriate amount of ashwagandha whole root powder. In some embodiments, ashwagandha whole root powder is present in an amount of about 0.25 g to about 6 g per serving of a mood support composition provided herein. For example, ashwagandha whole root powder can be present in an amount of about 0.25 g to about 1 g, about 0.25 g to about 2 g, about 0.25 g to about 3 g, about 0.25 g to about 4 g, about 0.25 g to about 5 g, about 1 g to about 2 g, about 1 g to about 3 g, about 1 g to about 4 g, about 1 g to about 5 g, about 1 g to about 6 g, about 2 g to about 3 g, about 2 g to about 4 g, about 2 g to about 5 g, about 2 g to about 6 g, about 3 g to about 4 g, about 3 g to about 5 g, about 3 g to about 6 g, about 4 g to about 5 g, about 4 g to about 6 g, or about 5 g to about 6 g per serving of a mood support composition provided herein.

In some embodiments, a mood support composition as provided herein includes an ashwagandha extract. In some embodiments, an ashwagandha extract is an extract from ashwagandha leaves and/or ashwagandha roots. In some embodiments, an ashwagandha extract is an ashwagandha root extract. For example, an ashwagandha root extract can be an extract where no ashwagandha leaves were used in producing the extract. In some embodiments, the proportions of compounds in an ashwagandha extract are similar to their proportions found naturally (e.g., in ashwagandha leaves and/or ashwagandha roots).

The mood support compositions provided herein can contain any appropriate amount of an ashwagandha extract. In some embodiments, an ashwagandha extract is present in an amount of at least about 100 mg per serving of a mood support composition provided herein (e.g., at least about 125 mg, at least about 160 mg, at least about 200 mg, at least about 300 mg, at least about 400 mg, at least about 500 mg, at least about 600 mg, or at least about 800 mg per serving of a mood support composition provided herein). In some embodiments, an ashwagandha extract is present in an amount of no more than about 2000 mg per serving of a mood support composition provided herein (e.g., no more than about 1800 mg, no more than about 1600 mg, no more than about 1400 mg, no more than about 1200 mg, no more than about 1000 mg, no more than about 800 mg, or no more than about 600 mg per serving of a mood support composition provided herein).

In some embodiments, an ashwagandha extract is present in an amount of about 125 mg to about 1800 mg per serving of a mood support composition provided herein. For example, an ashwagandha extract can be present in an amount of about 125 mg to about 200 mg, about 125 mg to about 400 mg, about 125 mg to about 600 mg, about 125 mg to about 800 mg, about 125 mg to about 1000 mg, about 125 mg to about 1200 mg, about 125 mg to about 1400 mg, about 125 mg to about 1600 mg, about 200 mg to about 400 mg, about 200 mg to about 600 mg, about 200 mg to about 800 mg, about 200 mg to about 1000 mg, about 200 mg to about 1200 mg, about 200 mg to about 1400 mg, about 200 mg to about 1600 mg, about 200 mg to about 1800 mg, about 400 mg to about 600 mg, about 400 mg to about 800 mg, about 400 mg to about 1000 mg, about 400 mg to about 1200 mg, about 400 mg to about 1400 mg, about 400 mg to about 1600 mg, about 400 mg to about 1800 mg, about 600 mg to about 800 mg, about 600 mg to about 1000 mg, about 600 mg to about 1200 mg, about 600 mg to about 1400 mg, about 600 mg to about 1600 mg, about 600 mg to about 1800 mg, about 800 mg to about 1000 mg, about 800 mg to about 1200 mg, about 800 mg to about 1400 mg, about 800 mg to about 1600 mg, about 800 mg to about 1800 mg, about 1000 mg to about 1200 mg, about 1000 mg to about 1400 mg, about 1000 mg to about 1600 mg, about 1000 mg to about 1800 mg, about 1200 mg to about 1400 mg, about 1200 mg to about 1600 mg, about 1200 mg to about 1800 mg, about 1400 mg to about 1600 mg, about 1400 mg to about 1800 mg, or about 1600 mg to about 1800 mg per serving of a mood support composition provided herein. In some embodiments, an ashwagandha extract is present in an amount of about 300 mg to about 675 mg per serving of a mood support composition provided herein. In some embodiments, an ashwagandha extract is present in an amount of about 525 mg to about 675 mg per serving of a mood support composition provided herein. In some embodiments, an ashwagandha extract is present in an amount of about 550 mg to about 650 mg per serving of a mood support composition provided herein.

In some embodiments, an ashwagandha extract is present in an amount of about 200 mg, 300 mg, about 400 mg, about 500 mg, about 525 mg, about 550 mg, about 575 mg, about 600 mg, about 625 mg, about 650 mg, about 675 mg, about 700 mg, about 800 mg, about 900 mg, about 1000 mg, about 1200 mg, about 1400 mg, about 1600 mg, or about 1800 mg per serving of a mood support composition provided herein. In some embodiments, an ashwagandha extract is present in an amount of about 600 mg per serving of a mood support composition provided herein.

In some embodiments, an ashwagandha extract is present in an amount of about 10% to about 95% w/w of a mood support composition provided herein. For example, an ashwagandha extract can be present in an amount of about 10% to about 20%, about 10% to about 30%, about 10% to about 40%, about 10% to about 50%, about 10% to about 60%, about 10% to about 70%, about 10% to about 80%, about 10% to about 90%, about 20% to about 30%, about 20% to about 40%, about 20% to about 50%, about 20% to about 60%, about 20% to about 70%, about 20% to about 80%, about 20% to about 90%, about 20% to about 95%, about 30% to about 40%, about 30% to about 50%, about 30% to about 60%, about 30% to about 70%, about 30% to about 80%, about 30% to about 90%, about 30% to about 95%, about 40% to about 50%, about 40% to about 60%, about 40% to about 70%, about 40% to about 80%, about 40% to about 90%, about 40% to about 95%, about 50% to about 60%, about 50% to about 70%, about 50% to about 80%, about 50% to about 90%, about 50% to about 95%, about 60% to about 70%, about 60% to about 80%, about 60% to about 90%, about 60% to about 95%, about 70% to about 80%, about 70% to about 90%, about 70% to about 95%, about 80% to about 90%, about 80% to about 95%, or about 90% to about 95% w/w of a mood support composition provided herein. In some embodiments, an ashwagandha extract is present in an amount of about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% w/w of a mood support composition provided herein.

Ashwagandha whole root powders and ashwagandha extracts can contain one or more withanolides. Withanolides include compounds consisting of a steroid backbone bound to a lactone or a lactone derivative. Non-limiting examples of a withanolide include withaferin A, salpichrolid A, nicandrenone-1, ixocarpalacton A, withaperuvin B, withangulatin C, acnistin I, physalin X, jaborosalactol 18, physalin C, jaborosalactone 7, salpichrolide G, jaborosalactone 37, jaborosalactone 43, subtrifloralactone E, tubonolide A, withametelinone, withaphysalin N, isocarpalactone B, perulactone C, jaborosalactone 15, subtrifloralactone K, taccalonolide A, jaborosalactol 24, physanolide A, exodeconolide A, withanoside VII, 27-hydroxywithanone, dihydrowithaferin A, viscosalactone B, withanoside IV, withanoside V, withaferin A, 12-deoxywithastramonolide, withanolide A, withanone, and withanolide B. In some embodiments, the one or more withanolides include withaferin A.

In some embodiments, ashwagandha whole root powder contains one or more withanolides present in an amount of at least about 0.4%, e.g., at least about 0.5%, at least about 0.6%, at least about 0.7%, at least about 0.8%, at least about 0.9%, at least about 1%, or at least about 1.2%, w/w of the ashwagandha whole root powder. In some embodiments, ashwagandha whole root powder contains one or more withanolides present in an amount of about 0.5% to about 1.5% w/w of the ashwagandha whole root powder. For example, ashwagandha whole root powder can contain one or more withanolides present in an amount of about 0.5% to about 0.8%, about 0.5% to about 1%, about 0.5% to about 1.2%, about 0.8% to about 1%, about 0.8% to about 1.2%, about 0.8% to about 1.5%, about 1% to about 1.2%, about 1% to about 1.5%, or about 1.2% to about 1.5% w/w of the ashwagandha whole root powder.

In some embodiments, an ashwagandha extract contains one or more withanolides present in an amount of at least about 4%, e.g., at least about 5%, at least about 6%, at least about 7%, at least about 8%, at least about 9%, or at least about 10% w/w of the ashwagandha extract. In some embodiments, the ashwagandha extract contains one or more withanolides present in an amount of about 1% to about 12% w/w of the ashwagandha extract. For example, the ashwagandha extract can contain one or more withanolides present in an amount of about 1% to about 2%, about 1% to about 3%, about 1% to about 4%, about 1% to about 5%, about 1% to about 6%, about 1% to about 7%, about 1% to about 8%, about 1% to about 9%, about 2% to about 3%, about 2% to about 4%, about 2% to about 5%, about 2% to about 6%, about 2% to about 7%, about 2% to about 8%, about 2% to about 9%, about 2% to about 10%, about 3% to about 4%, about 3% to about 5%, about 3% to about 6%, about 3% to about 7%, about 3% to about 8%, about 3% to about 9%, about 3% to about 10%, about 4% to about 5%, about 4% to about 6%, about 4% to about 7%, about 4% to about 8%, about 4% to about 9%, about 4% to about 10%, about 5% to about 6%, about 5% to about 7%, about 5% to about 8%, about 5% to about 9%, about 5% to about 10%, about 6% to about 7%, about 6% to about 8%, about 6% to about 9%, about 6% to about 10%, about 7% to about 8%, about 7% to about 9%, about 7% to about 10%, about 8% to about 9%, about 8% to about 10%, or about 9% to about 10% w/w of the ashwagandha extract. In some embodiments, the ashwagandha extract contains one or more withanolides present in an amount of about 5% w/w of the ashwagandha extract.

In some embodiments, the one or more withanolides are present in an amount of about 5 mg to about 100 mg per serving of a mood support composition provided herein. For example, the one or more withanolides are present in an amount of about 5 mg to about 10 mg, about 5 mg to 20 mg, about 5 mg to about 30 mg, about 5 mg to about 40 mg, about 5 mg to about 50 mg, about 5 mg to about 60 mg, about 5 mg to about 70 mg, about 5 mg to about 80 mg, about 5 mg to about 90 mg, about 10 mg to 20 mg, about 10 mg to about 30 mg, about 10 mg to about 40 mg, about 10 mg to about 50 mg, about 10 mg to about 60 mg, about 10 mg to about 70 mg, about 10 mg to about 80 mg, about 10 mg to about 90 mg, about 10 mg to about 100 mg, about 20 mg to about 30 mg, about 20 mg to about 40 mg, about 20 mg to about 50 mg, about 20 mg to about 60 mg, about 20 mg to about 70 mg, about 20 mg to about 80 mg, about 20 mg to about 90 mg, about 20 mg to about 100 mg, about 30 mg to about 40 mg, about 30 mg to about 50 mg, about 30 mg to about 60 mg, about 30 mg to about 70 mg, about 30 mg to about 80 mg, about 30 mg to about 90 mg, about 30 mg to about 100 mg, about 40 mg to about 50 mg, about 40 mg to about 60 mg, about 40 mg to about 70 mg, about 40 mg to about 80 mg, about 40 mg to about 90 mg, about 40 mg to about 100 mg, about 50 mg to about 60 mg, about 50 mg to about 70 mg, about 50 mg to about 80 mg, about 50 mg to about 90 mg, about 50 mg to about 100 mg, about 60 mg to about 70 mg, about 60 mg to about 80 mg, about 60 mg to about 90 mg, about 60 mg to about 100 mg, about 70 mg to about 80 mg, about 70 mg to about 90 mg, about 70 mg to about 100 mg, about 80 mg to about 90 mg, about 80 mg to about 100 mg, or about 90 mg to about 100 mg per serving of a mood support composition provided herein. In some embodiments, the one or more withanolides are present in an amount of about 5 mg, 10 mg, 20 mg, about 30 mg, about 40 mg, about 50 mg, about 60 mg, about 70 mg, about 80 mg, about 90 mg, or about 100 mg per serving of a mood support composition provided herein.

In some embodiments, the one or more withanolides are present in an amount of about 0.5% to about 10% w/w of a mood support composition provided herein. For example, the one or more withanolides can be present in an amount of about 0.5% to about 1%, about 0.5% to about 2%, about 0.5% to about 3%, about 0.5% to about 4%, about 0.5% to about 5%, about 0.5% to about 6%, about 0.5% to about 7%, about 0.5% to about 8%, about 0.5% to about 9%, about 1% to about 2%, about 1% to about 3%, about 1% to about 4%, about 1% to about 5%, about 1% to about 6%, about 1% to about 7%, about 1% to about 8%, about 1% to about 9%, about 1% to about 10%, about 2% to about 3%, about 2% to about 4%, about 2% to about 5%, about 2% to about 6%, about 2% to about 7%, about 2% to about 8%, about 2% to about 9%, about 2% to about 10%, about 3% to about 4%, about 3% to about 5%, about 3% to about 6%, about 3% to about 7%, about 3% to about 8%, about 3% to about 9%, about 3% to about 10%, about 4% to about 5%, about 4% to about 6%, about 4% to about 7%, about 4% to about 8%, about 4% to about 9%, about 4% to about 10%, about 5% to about 6%, about 5% to about 7%, about 5% to about 8%, about 5% to about 9%, about 5% to about 10%, about 6% to about 7%, about 6% to about 8%, about 6% to about 9%, about 6% to about 10%, about 7% to about 8%, about 7% to about 9%, about 7% to about 10%, about 8% to about 9%, about 8% to about 10%, or about 9% to about 10% w/w of a mood support composition provided herein. In some embodiments, the one or more withanolides are present in an amount of about 0.5%, about 1%, about 2%, about 3%, about 4%, about 5%, about 6%, about 7%, about 8%, about 9% or about 10% w/w of a mood support composition provided herein.

### Vitamin B12

The mood support compositions provided herein include vitamin B12. Certain B vitamins, including B12, can play a role in brain function and the production of neurotransmitters involved in mood regulation. Vitamin B12 includes hydroxocobalamin, cyanocobalamin, methylcobalamin, and adenosylcobalamin. Forms of vitamin B12 such as methylcobalamin, adenosylcobalamin, and hydroxycobalamin can have superior bioavailability compared to cyanocobalamin. See, e.g., Paul and Brady, Integr. Med. (Encinitas). 16(1): 42-49 (2017).

Any appropriate method can be used to obtain a vitamin B12 for use in the mood support compositions provided herein. For example, a vitamin B12 can be obtained by chemical synthesis.

The mood support compositions provided herein can contain any appropriate amount of vitamin B12. In some embodiments, vitamin B12 is present in an amount of at least about 0.4 μg per serving of a mood support composition provided herein (e.g., at least about 0.5 μg, at least about 1 μg, at least about 5 μg, at least about 10 μg, at least about 20 μg, at least about 30 μg, at least about 40 μg, at least about 50 μg, or at least about 60 μg per serving of a mood support composition provided herein). In some embodiments, vitamin B12 is present in an amount of no more than about 120 μg per serving of a mood support composition provided herein (e.g., no more than about 100 μg, no more than about 90 μg, no more than about 80 μg, no more than about 70 μg, no more than about 60 μg, no more than about 50 μg, no more than about 40 μg, no more than about 30 μg, or no more than about 20 μg per serving of a mood support composition provided herein).

In some embodiments, vitamin B12 is present in an amount of about 0.5 μg to about 100 μg per serving of a mood support composition provided herein. For example, vitamin B12 can be present in an amount of about 0.5 μg to about 1 μg, about 0.5 μg to about 5 μg, about 0.5 μg to about 10 μg, about 0.5 μg to about 20 μg, about 0.5 μg to about 30 μg, about 0.5 μg to about 40 μg, about 0.5 μg to about 50 μg, about 0.5 μg to about 60 μg, about 0.5 μg to about 70 μg, about 0.5 μg to about 80 μg, about 0.5 μg to about 90 μg, about 1 μg to about 5 μg, about 1 μg to about 10 μg, about 1 μg to about 20 μg, about 1 μg to about 30 μg, about 1 μg to about 40 μg, about 1 μg to about 50 μg, about 1 μg to about 60 μg, about 1 μg to about 70 μg, about 1 μg to about 80 μg, about 1 μg to about 90 μg, about 1 μg to about 100 μg, about 5 μg to about 10 μg, about 5 μg to about 20 μg, about 5 μg to about 30 μg, about 5 μg to about 40 μg, about 5 μg to about 50 μg, about 5 μg to about 60 μg, about 5 μg to about 70 μg, about 5 μg to about 80 μg, about 5 μg to about 90 μg, about 5 μg to about 100 μg, about 10 μg to about 20 μg, about 10 μg to about 30 μg, about 10 μg to about 40 μg, about 10 μg to about 50 μg, about 10 μg to about 60 μg, about 10 μg to about 70 μg, about 10 μg to about 80 μg, about 10 μg to about 90 μg, about 10 μg to about 100 μg, about 20 μg to about 30 μg, about 20 μg to about 40 μg, about 20 μg to about 50 μg, about 20 μg to about 60 μg, about 20 μg to about 70 μg, about 20 μg to about 80 μg, about 20 μg to about 90 μg, about 20 μg to about 100 μg, about 30 μg to about 40 μg, about 30 μg to about 50 μg, about 30 μg to about 60 μg, about 30 μg to about 70 μg, about 30 μg to about 80 μg, about 30 μg to about 90 μg, about 30 μg to about 100 μg, about 40 μg to about 50 μg, about 40 μg to about 60 μg, about 40 μg to about 70 μg, about 40 μg to about 80 μg, about 40 μg to about 90 μg, about 40 μg to about 100 μg, about 50 μg to about 60 μg, about 50 μg to about 70 μg, about 50 μg to about 80 μg, about 50 μg to about 90 μg, about 50 μg to about 100 μg, about 60 μg to about 70 μg, about 60 μg to about 80 μg, about 60 μg to about 90 μg, about 60 μg to about 100 μg, about 70 μg to about 80 μg, about 70 μg to about 90 μg, about 70 μg to about 100 μg, about 80 μg to about 90 μg, about 80 μg to about 100 μg, or about 90 μg to about 100 μg per serving of a mood support composition provided herein. In some embodiments, vitamin B12 is present in an amount of about 3 μg to about 60 μg per serving of a mood support composition provided herein.

In some embodiments, vitamin B12 is present in an amount of about 2 μg to about 10 μg per serving of a mood support composition provided herein. In some embodiments, vitamin B12 is present in an amount of about 4 μg to about 8 μg per serving of a mood support composition provided herein.

In some embodiments, vitamin B12 is present in an amount of 0.5 μg, 1 μg, about 2 μg, about 3 μg, about 4 μg, about 5 μg, about 6 μg, about 7 μg, about 8 μg, about 9 μg, about 10 μg, about 20 μg, about 30 μg, about 40 μg, about 50 μg, about 60 μg, about 80 μg, about 100 μg per serving of a mood support composition provided herein. In some embodiments, vitamin B12 is present in an amount of about 6 μg per serving of a mood support composition provided herein.

In some embodiments, vitamin B12 is present in an amount of about 0.0001% to about 0.5% w/w of a mood support composition provided herein. For example, vitamin B12 can be present in an amount of about 0.0001% to about 0.0005%, about 0.0001% to about 0.001%, about 0.0001% to about 0.005%, about 0.0001% to about 0.01%, about 0.0001% to about 0.05%, about 0.0001% to about 0.1%, about 0.0005% to about 0.001%, about 0.0005% to about 0.005%, about 0.0005% to about 0.01%, about 0.0005% to about 0.05%, about 0.0005% to about 0.1%, about 0.0005% to about 0.5%, about 0.001% to about 0.005%, about 0.001% to about 0.01%, about 0.001% to about 0.05%, about 0.001% to about 0.1%, about 0.001% to about 0.5%, about 0.005% to about 0.01%, about 0.005% to about 0.05%, about 0.005% to about 0.1%, about 0.005% to about 0.5%, about 0.01% to about 0.05%, about 0.01% to about 0.1%, about 0.01% to about 0.5%, about 0.05% to about 0.1%, about 0.05% to about 0.5%, or about 0.1% to about 0.5% w/w of a mood support composition provided herein. In some embodiments, vitamin B12 is present in an amount of about 0.0001%, about 0.0005%, about 0.001%, about 0.005%, about 0.01%, about 0.05%, about 0.1%, about 0.5% w/w of a mood support composition provided herein.

In some embodiments, the vitamin B12 is methylcobalamin.

### Folate

The mood support compositions provided herein can further include folate. Folate (also known as vitamin B9) refers to folates naturally found in foods and folates used in dietary supplements and fortified foods such as folic acid. Non-limiting examples of folate include folic acid, tetrahydrofolic acid, 5-methyltetrahydrofolate (also known as 5-MTHF, 5-methyl-folate, and methylfolate) or a salt thereof, a methenyltetrahydrofolate, folinic acid, folacin, and pteroylglutamic acid. Non-limiting examples of salts of 5-methyltetrahydrofolate include calcium L-5-methyltetrahydrofolate and sodium L-5-methyltetrahydrofolate.

Any appropriate method can be used to obtain folate for use in the mood support compositions provided herein. For example, folates can be obtained by chemical synthesis.

The mood support compositions provided herein can contain any appropriate amount of folate. In some embodiments, folate is present in an amount of at least about 0.05 mg per serving of a mood support composition provided herein (e.g., at least about 0.1 mg, at least about 0.2 mg, at least about 0.4 mg, at least about 0.6 mg, at least about 0.8 mg, at least about 1 mg, at least about 1.4 mg, or at least about 1.6 mg per serving of a mood support composition provided herein). In some embodiments, folate is present in an amount of no more than about 2.2 mg per serving of a mood support composition provided herein (e.g., no more than about 2 mg, no more than about 1.8 mg, no more than about 1.6 mg, no more than about 1.4 mg, no more than about 1 mg, no more than about 0.8 mg, no more than about 0.6 mg, or no more than about 0.4 mg per serving of a mood support composition provided herein).

In some embodiments, folate is present in an amount of about 0.1 mg to about 2 mg per serving of a mood support composition provided herein. For example, folate can be present in an amount of about 0.1 mg to about 0.2 mg, about 0.1 mg to about 0.4 mg, about 0.1 mg to about 0.6 mg, about 0.1 mg to about 0.8 mg, about 0.1 mg to about 1 mg, about 0.1 mg to about 1.2 mg, about 0.1 mg to about 1.4 mg, about 0.1 mg to about 1.6 mg, about 0.1 mg to about 1.8 mg, about 0.2 mg to about 0.4 mg, about 0.2 mg to about 0.6 mg, about 0.2 mg to about 0.8 mg, about 0.2 mg to about 1 mg, about 0.2 mg to about 1.2 mg, about 0.2 mg to about 1.4 mg, about 0.2 mg to about 1.6 mg, about 0.2 mg to about 1.8 mg, about 0.2 mg to about 2 mg, about 0.4 mg to about 0.6 mg, about 0.4 mg to about 0.8 mg, about 0.4 mg to about 1 mg, about 0.4 mg to about 1.2 mg, about 0.4 mg to about 1.4 mg, about 0.4 mg to about 1.6 mg, about 0.4 mg to about 1.8 mg, about 0.4 mg to about 2 mg, about 0.6 mg to about 0.8 mg, about 0.6 mg to about 1 mg, about 0.6 mg to about 1.2 mg, about 0.6 mg to about 1.4 mg, about 0.6 mg to about 1.6 mg, about 0.6 mg to about 1.8 mg, about 0.6 mg to about 2 mg, about 0.8 mg to about 1 mg, about 0.8 mg to about 1.2 mg, about 0.8 mg to about 1.4 mg, about 0.8 mg to about 1.6 mg, about 0.8 mg to about 1.8 mg, about 0.8 mg to about 2 mg, about 1 mg to about 1.2 mg, about 1 mg to about 1.4 mg, about 1 mg to about 1.6 mg, about 1 mg to about 1.8 mg, about 1 mg to about 2 mg, about 1.2 mg to about 1.4 mg, about 1.2 mg to about 1.6 mg, about 1.2 mg to about 1.8 mg, about 1.2 mg to about 2 mg, about 1.4 mg to about 1.6 mg, about 1.4 mg to about 1.4 mg, about 1.2 mg to about 2 mg, about 1.6 mg to about 1.4 mg, about 1.6 mg to about 2 mg, or about 1.8 mg to about 2 mg per serving of a mood support composition provided herein. In some embodiments, folate is present in an amount of about 0.2 mg to about 0.8 mg per serving of a mood support composition provided herein.

In some embodiments, folate is present in an amount of about 0.2 mg, about 0.4 mg, about 0.6 mg, about 0.8 mg, about 1 mg, about 1.4 mg, or about 2 mg per serving of a mood support composition provided herein. In some embodiments, folate is present in an amount of about 0.4 mg per serving of a mood support composition provided herein.

In some embodiments, the amount of folate in mg is the amount of folate as mg of dietary folate equivalents (DFEs). In some embodiments, 1 mg DFE is equal to 1 mg food folate. In some embodiments, 1 mg DFE is equal to 0.6 mg folic acid from fortified foods or dietary supplements consumed with foods. In some embodiments, 1 mg DFE is equal to 0.5 mg folic acid from dietary supplements taken on an empty stomach. In some embodiments, 1 mg DFE is equal to 0.59 mg of a methyltetrahydrofolate. For example, if a mood support composition provided herein contains 0.4 mg DFE of a methyltetrahydrofolate, it contains 0.235 mg of the methyltetrahydrofolate.

In some embodiments, folate is present in an amount of about 0.001% to about 1% w/w of a mood support composition provided herein. For example, folate can be present in an amount of about 0.001% to about 0.005%, about 0.001% to about 0.01%, about 0.001% to about 0.05%, about 0.001% to about 0.1%, about 0.001% to about 0.5%, about 0.005% to about 0.01%, about 0.005% to about 0.05%, about 0.005% to about 0.1%, about 0.005% to about 0.5%, about 0.005% to about 1%, about 0.01% to about 0.05%, about 0.01% to about 0.1%, about 0.01% to about 0.5%, about 0.01% to about 1%, about 0.05% to about 0.1%, about 0.05% to about 0.5%, about 0.05% to about 1%, about 0.1% to about 0.5%, about 0.1% to about 1%, or about 0.5% to about 1% w/w of a mood support composition provided herein. In some embodiments, folate is present in an amount of about 0.001%, about 0.005%, about 0.01%, about 0.05%, about 0.1%, about 0.5%, or about 1% w/w of a mood support composition provided herein.

In some embodiments, the folate is calcium methyltetrahydrofolate.

In some embodiments, a mood support composition provided herein is a composition that includes the ingredients listed in Table 1.

TABLE 1

| | Amount |
|---|---|
| Ingredient | (per serving) |
| Saffron extract | 28 mg to 30 mg |
| Ashwagandha extract | 300 mg to 675 mg |
| Vitamin B12 | 3 μg to 60 μg |
| Folate | 200 μg to 800 μg DFE |

In some embodiments, the ashwangdha extract contains at least about 5% withanolides. In some embodiments, the ashwangdha extract contains about 5% withanolides. In some embodiments, the ashwagandha extract is an ashwagandha root extract. In some embodiments, the vitamin B12 is methylcobalamin. In some embodiments, the folate is calcium methyltetrahydrofolate.

In some embodiments, a mood support composition provided herein is a composition that includes the ingredients listed in Table 2.

TABLE 2

| | Amount | |
|---|---|---|
| Ingredient | (per serving) | |
| Saffron extract | 28 | mg |
| Ashwagandha extract | 600 | mg |
| Vitamin B12 | 6 | μg |
| Folate | 400 | μg DFE |

In some embodiments, the ashwangdha extract contains at least about 5% withanolides. In some embodiments, the ashwangdha extract contains about 5% withanolides. In some embodiments, the ashwagandha extract is an ashwagandha root extract. In some embodiments, the vitamin B12 is methylcobalamin. In some embodiments, the folate is calcium methyltetrahydrofolate.

A mood support composition provided herein can be formulated for oral use and can further include one or more suitable excipients, sweetening agents, flavorants, colorants, preservatives, and/or other ingredients. Non-limiting examples of oral formulations include tablets, troches, lozenges, aqueous or oil suspensions, non-aqueous solutions, dispersible powders or granules (including micronized particles or nanoparticles), emulsions, hard or soft capsules, syrups, and elixirs. Compositions intended for oral use can be prepared according to any appropriate method, including standard methods for the manufacture of pharmaceutical compositions. Preparations for oral use can be suitably formulated to give controlled release of one or more compounds.

Liquid preparations for oral administration can take the form of, for example, solutions, syrups, or suspension, or they can be presented as a dry product for reconstitution with saline or other suitable liquid vehicle before use. In some embodiments, liquid preparations can contain pharmaceutically acceptable additives such as suspending agents, emulsifying agents, non-aqueous vehicles, preservatives, buffer salts, flavoring agents, coloring agents, and sweetening agents as appropriate.

Tablets, capsules, or soft gels can be prepared with one or more suitable excipients, such as binding agents, fillers, lubricants, disintegrants, and/or wetting agents. In some embodiments, a capsule, tablet, or soft gel form of the mood support compositions provided herein further includes one or more of microcrystalline cellulose, an amorphous silica, silicon dioxide, magnesium stearate, and hydroxypropyl methylcellulose. In some embodiments, a capsule, tablet, or soft gel form of the mood support compositions provided herein further includes one or more of microcrystalline cellulose, magnesium stearate, and hydroxypropyl methylcellulose.

In some embodiments, a tablet of a mood support composition provided herein can have a coating (e.g., a polymer or polysaccharide-based coating with or without plasticizers and/or pigments). Such coatings can include, for example, one or more of a film-forming polymer, a colorant, and a flavorant. Non-limiting examples of a flavorant include vanillin, vanilla extract, artificial vanilla flavorant, and natural caramel. In some embodiments, a mood support composition can have a vanilla or vanilla caramel flavor profile. In some embodiments, a coating of a tablet of a mood support composition provided herein includes one or more of: hydroxypropyl methyl cellulose, sodium alginate, calcium carbonate, calcium sulfate, magnesium carbonate, cellulose gum, isomalt, polyethylene glycol, glycerin, hydroxypropyl cellulose, and carnauba wax.

In some embodiments, a capsule, tablet, or soft gel form of the mood support compositions provided herein can have a unit dosage equal to the daily desired dosage for a particular mammal. For example, if it is desired to administer 100 mg of a particular agent to a mammal, each tablet can include about 100 mg in weight of that agent. As used herein, mammals generally refer to humans, but also can include domesticated mammals (e.g., dogs, cats, and livestock such as cows, horses, pigs, or sheep). The dosages of a particular mood support composition provided herein will depend on many factors, including the general health of a mammal. In some cases, a total daily dose (e.g., a full serving) can be prepared and administered in the form of one or more dosage units (e.g., one tablet, capsule, or soft gel, two tablets, capsules, or soft gels, three tablets, capsules, or soft gels, four tablets, capsules or soft gels, five tablets, capsules, or soft gels, or six tablets, capsules, or soft gels). For instance, in some cases, one tablet, one capsule, or one soft gel can include a full serving (e.g., daily dosage) of the composition. In some cases, two tablets, two capsules, or two soft gels in combination can include a full serving (e.g., daily dosage) of the composition. In some cases, three tablets, three capsules, or three soft gels in combination can include a full serving (e.g., daily dosage) of the composition.

In some embodiments, a mood support composition provided herein can be substantially free of artificial colors, artificial flavors, and chemical preservatives.

In some embodiments, a mood support composition provided herein can be a composition that includes ingredients as shown in Table 3.

TABLE 3

| | |
|---|---|
| | Amount |
| Ingredient | (per serving) |

| | | |
|---|---|---|
| Saffron extract | 28 | mg |
| Ashwagandha extract | 600 | mg |
| Vitamin B12 | 6 | μg |
| Folate | 400 | μg DFE |
| Binding agent(s), filler(s), lubricant(s), | 153 | mg |
| disintegrant(s), colorant(s), and flavorant(s) | | |

In some embodiments, the ashwangdha extract contains at least about 5% withanolides. In some embodiments, the ashwangdha extract contains about 5% withanolides. In some embodiments, the ashwagandha extract is an ashwagandha root extract. In some embodiments, the vitamin B12 is methylcobalamin. In some embodiments, the folate is calcium methyltetrahydrofolate.

### Methods

The mood support compositions provided herein can be useful for one or more of supporting a positive mental outlook, better coping with the ups and downs of life, maintaining a balanced mood, promoting emotional health, supporting emotional balance, supporting the activities of brain messengers, supporting general well-being, supporting more restful sleep (e.g., restful sleep without sedation), reducing feelings of increased sadness, reducing feelings of occasional stress, supporting mood-related sexual health, and promoting physical and mental energy. For example, a mammal may feel less stressed, feel more positive, and/or feel more energetic after consuming a mood support composition provided herein as compared to a day where a mood support composition provided herein was not consumed.

Provided herein are methods of improving one or more aspects related to mood in a mammal, comprising administering to the mammal any of the compositions described herein. In some embodiments, improving an aspect related to mood comprises one or more of improving emotional health, improving the ability to cope with stress, improving the activity of a brain messenger, improving emotional balance, improving general well-being, improving the restfulness of sleep, and improving physical and mental energy.

Also provided herein are methods of reducing feelings of sadness and/or feelings of stress in a mammal, comprising administering to the mammal any of the compositions described herein.

Aspects related to mood can be assessed using any appropriate method(s). Non-limiting examples of methods that can be used to assess one or more of a positive mental outlook, coping with the ups and downs of life, a balanced mood, emotional health, emotional balance, general well-being, more restful sleep (e.g., restful sleep without sedation), feelings of increased sadness, feelings of occasional stress, mood-related sexual health, physical energy, and mental energy mood include HAM-D, HAM-A Mental Fatigue, Alertness, and Motivation Visual Analogue Scales (VAS), Mental and Physical State Energy and Fatigue Scales, Bond-Lader Scale, and Mood, Alertness, and Physical Sensations Scales (MAPPS). See e.g., Reed et al. J. Cogn. Enhanc. 3:193-206 (2019); Tartar et al. Cureus 13 (2021).

HAM-D and HAM-A are clinician-administered depression assessment and anxiety assessment scales, respectively. See, e.g., Rohan, et al. J Affect Disord. 200: 111-118 (2016); and Lopresti et al. Medicine (Baltimore). 98(37): e17186 (2019). Mental Fatigue, Alertness, and Motivation Visual Analogue Scales can be used to assess current feelings of mental fatigue, alertness, and motivation. For example, the mental fatigue and alertness scales can be scored from “not at all” to “extremely,” while the motivation scale can be scored from “no motivation at all” to “strongest degree of motivation imaginable,” with scores ranging from 0 to 100. Mental and Physical State Energy and Fatigue Scales (EFS-State Scales) can include reporting of a mammal's (e.g., a human) current subjective feelings of physical energy, physical fatigue, mental energy, and mental fatigue. In some embodiments, current subjective feelings can be reported by selecting a point along a visual analog scale for three items for each EFS-State scale dimension. For example, energy items can include (i) “I feel I have no energy” to “strongest feelings of energy ever felt”; (ii) “I feel I have no vigor” to “strongest feelings of vigor ever felt”; and (iii) “I feel I have no pep” to “strongest feelings of pep ever felt”; fatigue items can include (i) “I feel no fatigue” to “strongest feelings of fatigue ever felt”; (ii) “I feel no exhaustion” to “strongest feelings of exhaustion ever felt”; and (iii) “I have no feelings of being worn out” to “strongest feelings of being worn out ever felt.” The Bond-Lader Scale is derived from 16 scales that are anchored on each side by adjectives describing a mood and can provide three composite scores for Alert, Calm, and Content. See e.g., Reed et al. J Cogn. Enhanc. 3:193-206 (2019).

In some embodiments, an improvement in an aspect related to occurs within about 1 day to about 8 weeks of consuming a mood composition as provided herein. For example, within about 1 day to about 1 week, about 1 day to about 2 weeks, about 1 day to about 3 weeks, about 1 day to about 4 weeks, about 1 day to about 6 weeks, about 1 week to about 2 weeks, about 1 week to about 3 weeks, about 1 week to about 4 weeks, about 1 week to about 6 weeks, about 1 week to about 8 weeks, about 2 weeks to about 3 weeks, about 2 weeks to about 4 weeks, about 2 weeks to about 6 weeks, about 2 weeks to about 8 weeks, about 3 weeks to about 4 weeks, about 3 weeks to about 6 weeks, about 3 weeks to about 8 weeks, about 4 weeks to about 6 weeks, about 4 weeks to about 8 weeks, or about 6 weeks to about 8 weeks of consuming a mood composition as provided herein. In some embodiments, the improvement in an aspect related to occurs within about 2 weeks to about 4 weeks of consuming a mood composition as provided herein.

The frequency of consuming a mood support composition provided herein can vary from once or twice a week to once or more times a day, such as two or three times daily. In some embodiments, a mood support composition provided herein is consumed once daily. The frequency of consuming a mood support composition provided herein can be constant or be variable during the duration of the treatment.

In some embodiments, wherein a total daily dose (e.g., a full serving) of a mood support composition provided herein is consumed in the form of two or more dosage units, at least one of the dosage units is consumed at a different time than at least one other dosage unit. For example, when two tablets, capsules, or soft gels together contain a full serving of a mood support composition provided herein, one capsule is consumed in the morning and another capsule is consumed in the evening.

### Exemplary Embodiments

An embodiment described herein provides a mood support composition. The mood support composition includes a saffron extract, an ashwagandha extract, a folate, vitamin B12, where the vitamin B12 includes methylcobalamin.

In an embodiment, combinable with any other embodiment, the saffron extract is present in an amount of about 14 mg to about 56 mg per serving of the mood support composition.

In an embodiment, combinable with any other embodiment, the saffron extract is present in an amount of about 28 mg to about 30 mg per serving of the mood support composition.

In an embodiment, combinable with any other embodiment, the saffron extract is present in an amount of about 28 mg per serving of the mood support composition.

In an embodiment, combinable with any other embodiment, the saffron extract is an extract of the stigmas of Crocus sativus flowers.

In an embodiment, combinable with any other embodiment, the ashwagandha extract is present in an amount of about 125 mg to about 1800 mg per serving of the mood support composition.

In an embodiment, combinable with any other embodiment, the ashwagandha extract is present in an amount of about 300 mg to about 675 mg per serving of the mood support composition.

In an embodiment, combinable with any other embodiment, the ashwagandha extract is present in an amount of about 600 mg per serving of the mood support composition.

In an embodiment, combinable with any other embodiment, the ashwagandha extract contains one or more withanolides.

In an embodiment, combinable with any other embodiment, the ashwagandha extract is an ashwagandha root extract.

In an embodiment, combinable with any other embodiment, vitamin B12 is present in an amount of about 0.5 μg to about 100 μg per serving of the mood support composition.

In an embodiment, combinable with any other embodiment, vitamin B12 is present in an amount of about 3 μg to about 60 μg per serving of the mood support composition.

In an embodiment, combinable with any other embodiment, vitamin B12 is present in an amount of about 6 μg per serving of the mood support composition.

In an embodiment, combinable with any other embodiment, the folate is present in an amount of about 0.1 mg to about 2 mg DFE per serving of the mood support composition.

In an embodiment, combinable with any other embodiment, the folate is present in an amount of about 0.2 mg to about 0.8 mg DFE per serving of the mood support composition.

In an embodiment, combinable with any other embodiment, the folate is present in an amount of about 0.4 mg DFE per serving of the mood support composition.

In an embodiment, combinable with any other embodiment, the folate comprises calcium methyltetrahydrofolate.

In an embodiment, combinable with any other embodiment, the mood support composition is formulated as a tablet, a capsule, or a soft gel.

In an embodiment, combinable with any other embodiment, two tablets, capsules, or soft gels comprises a full serving of the mood support composition.

In an embodiment, combinable with any other embodiment, the mood support composition is formulated as a capsule.

A method of improving one or more of emotional health, the ability to cope with stress, the activity of a brain messenger, emotional balance, general well-being, the restfulness of sleep, physical energy, and mental energy in a mammal, the method including administering to the mammal the mood support composition of any other embodiment.

A method of reducing feelings of sadness and/or feelings of stress in a mammal, the method including administering to the mammal the mood support composition of any other embodiment.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A mood support composition comprising:
a saffron extract;
an ashwagandha extract;
a folate; and
vitamin B12, wherein the vitamin B12 comprises methylcobalamin.

2. The mood support composition of claim 1, wherein the saffron extract is present in an amount of about 14 mg to about 56 mg per serving of the mood support composition.

3. The mood support composition of claim 1, wherein the saffron extract is present in an amount of about 28 mg to about 30 mg per serving of the mood support composition.

4. The mood support composition of claim 1, wherein the saffron extract is present in an amount of about 28 mg per serving of the mood support composition.

5. The mood support composition of claim 1, wherein the saffron extract is an extract of the stigmas of Crocus sativus flowers.

6. The mood support composition of claim 1, wherein the ashwagandha extract is present in an amount of about 125 mg to about 1800 mg per serving of the mood support composition.

7. The mood support composition of claim 1, wherein the ashwagandha extract is present in an amount of about 300 mg to about 675 mg per serving of the mood support composition.

8. The mood support composition of claim 1, wherein the ashwagandha extract is present in an amount of about 600 mg per serving of the mood support composition.

9. The mood support composition of claim 1, wherein the ashwagandha extract contains one or more withanolides.

10. The mood support composition of claim 1, wherein the ashwagandha extract is an ashwagandha root extract.

11. The mood support composition of claim 1, wherein vitamin B12 is present in an amount of about 0.5 μg to about 100 μg per serving of the mood support composition.

12. The mood support composition of claim 1, wherein vitamin B12 is present in an amount of about 3 μg to about 60 μg per serving of the mood support composition.

13. The mood support composition of claim 1, wherein the folate is present in an amount of about 0.1 mg to about 2 mg DFE per serving of the mood support composition.

14. The mood support composition of claim 1, wherein the folate is present in an amount of about 0.2 mg to about 0.8 mg DFE per serving of the mood support composition.

15. The mood support composition of claim 1, wherein the folate comprises calcium methyltetrahydrofolate.

16. The mood support composition of claim 1, wherein the mood support composition is formulated as a tablet, a capsule, or a soft gel.

17. The mood support composition of claim 1, wherein two tablets, capsules, or soft gels comprises a full serving of the mood support composition.

18. The mood support composition of claim 1, wherein the mood support composition is formulated as a capsule.

19. A method of improving one or more of emotional health, the ability to cope with stress, the activity of a brain messenger, emotional balance, general well-being, the restfulness of sleep, physical energy, and mental energy in a mammal, comprising administering to the mammal a mood support composition of claim 1.

20. A method of reducing feelings of sadness and/or feelings of stress in a mammal, comprising administering to the mammal a mood support composition of claim 1.
